(19) United States
(12) Patent Application Publication
(10) Pub. No.: US 2025/0302451 A1
(43) Pub. Date: Oct. 2, 2025

(54) Fertility Kit

(71) Applicant: PULSENMORE LTD., Omer (IL)

(72) Inventors: Elazar SONNENSCHEIN, Omer (IL); Ravit LEVY, Tel Aviv-Yafo (IL); Minelu SONNENSCHEIN, Meitar (IL)

(21) Appl. No.: 19/089,757

(22) Filed: Mar. 25, 2025

(30) Foreign Application Priority Data
Apr. 1, 2024 (IL) 311869

(51) Int. Cl. A61B 10/00; A61B 8/00; A61B 8/08; A61B 17/435; G16H 10/60; G16H 20/10; G16H 40/60

(52) U.S. Cl. CPC A61B 10/0012; A61B 8/0866; A61B 8/565; A61B 10/0045; A61B 17/435; G16H 10/60; G16H 20/10; G16H 40/60

(57) ABSTRACT

A fertility kit comprises a fertility drug and dedicated ultrasound apparatus adapted to monitor the development of a patient's follicles, wherein the apparatus comprises a timing component suitable to suggest personalized monitoring times.

### FIELD OF THE INVENTION

The present invention relates to methods for in-vitro fertilization and egg preservation. More particularly, the invention relates to systems combining drugs useful in the IVF process together with dedicated ultrasound apparatus.

### BACKGROUND OF THE INVENTION

For women undergoing infertility treatment, administering injections (either subcutaneous or intramuscular) is a common occurrence. While clomiphene (Clomid) is taken orally, many other fertility drugs require multiple injections over several days or weeks. These injectable treatments are hormonal and aim to regulate hormone production or induce ovulation. Each drug has subtle differences in its mechanism of action, but ultimately, they all support fertility in some way.

The following are some commonly used fertility drugs:

hMG, or human menopausal gonadotropin, contains two human hormones: follicle-stimulating hormone (FSH) and luteinizing hormone (LH). Gonadotropins are commonly administered to women undergoing assisted reproduction technology treatments with the goal of stimulating the ovaries to produce multiple follicles (eggs) during one cycle. This drug is typically injected daily for 7 to 12 days in the first half of the menstrual cycle. FSH may also be given on its own for the same purpose and in a similar manner as hMG.

Human chorionic gonadotropin, or hCG, is a hormone used to trigger the release of eggs from the follicles (ovulation). It is given in combination with other fertility drugs that stimulate follicle production and is administered at a precise time during the menstrual cycle based on the results of blood tests and ultrasound examinations. It is the same hormone produced by the placenta during pregnancy and measured in pregnancy tests.

Gonadotropin Releasing Hormone (GnRH) agonists help regulate hormone levels during fertility treatment by shutting down the body's natural hormone production. This allows for precise control of egg development and the use of gonadotropin drugs to stimulate follicle production. GnRH agonists offer many advantages during fertility treatment. Gonadotropin Releasing Hormone Antagonist or GnRH antagonists have a similar effect as GnRH agonists, but rather than initially stimulating and then down-regulating ovarian hormone production, GnRH antagonists work by immediately blocking the release of ovarian hormones. Typically, fewer injections of GnRH antagonists are required because of their stronger effect in reducing ovarian hormone production.

A commonly used IVF protocol is called “down-regulation” or “long Lupron.” With this regimen, Lupron (leuprolide acetate) administration is started on about day 21 of the woman's cycle to “down-regulate” the pituitary gland with regard to the production of FSH and LH hormones. Lupron is given as a subcutaneous injection.

The next medication begins a few days after the menstrual period starts. FSH is administered to stimulate the ovaries to make multiple follicles and eggs. Once the FSH stimulating injections have begun, blood and ultrasound testing is done about every 2-3 days to monitor the development of the follicles in the ovaries and the hormone levels. Once a woman's follicles are mature and abundant, the eggs are carefully extracted from her ovaries through an egg aspiration procedure. These eggs are then fertilized with her partner's sperm in a laboratory and ultimately transferred back into her uterus for embryo implantation.

To monitor the effects of the fertility drugs, patients undergo multiple ultrasounds during the stage of treatment. Typically, the ultrasounds start three days after the fertility medication injections have started; follow-up ultrasounds are scheduled every one to two days. These ultrasounds provide information that allows the treating physician to either advance to the next stage of treatment or make changes to the fertility medication.

The same process is undergone by women desiring to undergo egg preservation, which involves harvesting the eggs when ready, and preserving them for future fertilization and implantation. After eggs are harvested, they are put into a deep freeze, and stored in liquid nitrogen tanks.

Some people's ovaries respond faster than others to stimulation, meaning follicles may mature at different rates. Accordingly, adhering to standard protocols may not provide the optimal timing for ultrasound checks. More so since different drugs have different stimulation times and require different monitoring schedules. Common drugs like follicle-stimulating hormone (FSH) typically require more frequent monitoring than others.

The need to visit a clinic so frequently during this already hard fertility treatment imposes an additional burden on the patient and on the clinic. It is also a cause of anxiety, which is harmful to the process. It would therefore be highly desirable to provide a way by which the patient is capable of providing ultrasound monitoring data to the healthcare professional who manages the IVF process by herself, just like she self-medicates with the fertility hormones during the process, without the need for the frequent visits to the clinic.

It is an object of the present invention to provide a fertility kit that allows patients undergoing IVF or egg preservation procedures to effectively monitor the progress of the treatment from the privacy and convenience of their homes. The fertility kit of the invention allows a personalized monitoring schedule that takes into account the specific drug used as well as the personal patient response to it. In the context of this specification the term “fertility kit” or “fertility” in general also apply to the egg preservation process, in which the eggs are not fertilized before freezing, as well as to processes in which at least some of the eggs are frozen after being fertilized.

It is a further object of the invention to provide a system comprising one or more fertility drugs together with ultrasound apparatus adapted for self-care by the patient.

It is yet another object of invention to provide a combination of one or more fertility drugs and ultrasound apparatus which are programmed such that they provide an increased monitoring precision and hence a better chance of success of the treatment.

### SUMMARY OF THE INVENTION

The invention relates to a fertility kit comprising a fertility drug and dedicated ultrasound apparatus, adapted to monitor the development of a patient's follicles, wherein the apparatus comprises a timing component suitable to suggest personalized monitoring times.

In some embodiments, the fertility kit of the invention further comprises ancillary tests suitable to provide information regarding the ovulation status of a subject, such as, for example, saliva and urine tests.

The invention further embodies an application adapted to run on a smart device, associated with the kit of the invention, wherein the smart device is suitable to be in communication with the dedicated ultrasound device.

Also encompassed by the invention is a system for improving the precision of the timing of ultrasound scans during an IVF preparatory process or an egg preservation process, comprising:
a) a fertility kit comprising a fertility drug and dedicated ultrasound apparatus;
b) an application adapted to run on a smart device and to be in communication with the ultrasound apparatus; and
c) a smart device capable of exchanging data with an ultrasound device and with remote computer systems, such computer systems located in the cloud in the cloud or in a health professional location.

### BRIEF DESCRIPTION OF THE DRAWINGS

In the drawings:

FIG. 1 shows an ultrasound scan of the ovaries of a subject;

FIG. 2 (A and B) are perspective views of a wireless device according to an embodiment of the invention; and

FIG. 3 schematically illustrates a kit according to the invention.

### DETAILED DESCRIPTION OF THE INVENTION

A system or a kit according to the invention comprises one or more fertility drug(s) that is injectable by a patient, and a personal ultrasound device specifically adapted to match the requirements of the IVF process. As will be further discussed below, the ultrasound device is associated with data specifically relevant to the drug or drugs included in the kit, thus improving the results of the self-monitoring process.

The ultrasound device can be, for instance, a device as described in PCT Patent Publication WO2023026272, which has been modified for the purposes of this invention. Specifically, one modification is such that the device of the invention comprises a timing component adapted to suggest to the patient when to perform the next ultrasound monitoring. The timing component obtains the suggested timing using a combination of a) the known behavior of the specific fertility drug contained in the kit; and b) the result(s) of the previous US scan(s). The timing component may be part of the processor that regulates the operation of the device, or can be a separate component. The processor may be configured to determine a time when the patient should perform the next scan taking into account the nature of the drugs administered to the patient and their known average effect on the timing of ovulation, the specific physical parameters of the patient that may affect the response to said drugs, and the history of the patient's response to said or other drugs, obtained from previous scans or previous cycles of attempted ovulation.

Moreover, the processor may have access to online data either from its connection to a smart device equipped with wi-fi or other communication ability or directly using network connectivity provided in the ultrasound device itself.

The timing component may alert the patient via a connection to a smart device, such as a smartphone equipped with a suitable app, or using any visual or audible indication located on the device itself, such as a light or a sound.

The ultrasound device can produce 2D, 3D or 4D images.
A 2D ultrasound apparatus is the standard ultrasound system found in clinics and hospitals. The ultrasound image is generated by transmitted acoustic waves at a megahertz frequency. The reflected waves are processed and generate the image. This process creates simple, black-and-white images that create a cross-sectional view, with bright spots for denser materials like bone.
3D ultrasound apparatus uses the same basic idea as 2D ultrasound but takes many images from different angles and processes them together to create an image that looks like a real photograph. In addition, it is possible to color the follicles with the same color (representing the same size) or to annotate them with characters/figures to represent the left/right ovary.
4D ultrasound apparatus adds a fourth dimension-time. In a 4D ultrasound, a series of 3D images is put together to form a low-resolution video.

The results of the scans can be transmitted by the ultrasound device to either a nearby smart device, or the cloud, or another computer in the clinic/hospital, or both, where the evaluation of the aforementioned data (i.e., the drug-specific expected behavior and the results from one or more previous scans) are analyzed. The result of this analysis determines how much and in which direction the next scanning time should diverge from the standard monitoring protocol, which information can be relayed to the timing component and used by the processor to provide a time alert to the user.

As will be apparent to the skilled person, the personalization of the procedure afforded by the kit of the invention can significantly contribute to the success of the IVF or egg preservation procedure and undoubtedly contributes to a less stressful and more pleasant experience for the patient.

In embodiments of the invention, additional components may be added to the kit, such as a saliva or urine testing component, which may add additional data that permits providing a more precise timing for the next ultrasound scan. In embodiments of the invention, the result of the test, such as the saliva or urine test, can be input into a dedicated app that is part of the system using the kit. The results can be analyzed locally in the timing component's processor or updated to the cloud for integration with the additional data from which a determination of the next desirable ultrasound scan timing is determined.

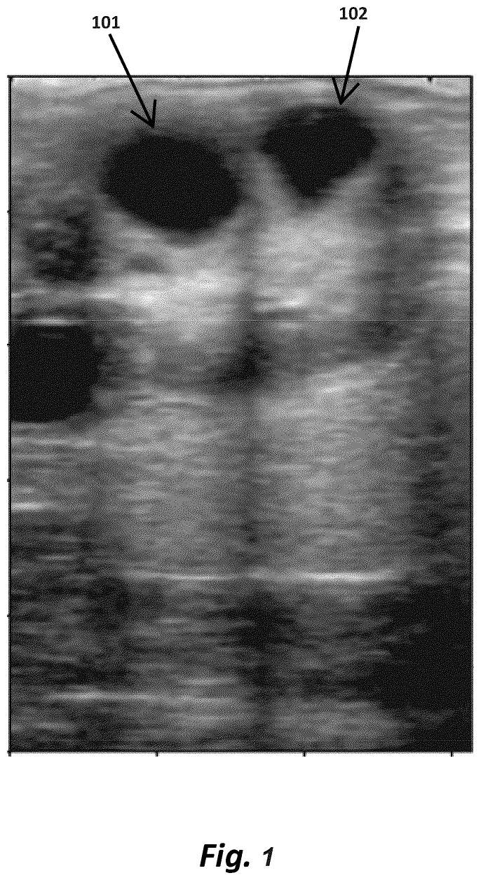

Reference is made to the aforementioned WO2023026272, which describes in detail the detection and analysis of the follicles and endometrium status using a device that operates, in that respect, like the device of the invention. For the sake of brevity, all the description of the imaging and analyzing procedures described in said publication is incorporated herein by reference. In brief, using the device of the invention, the ovaries can be seen as illustrated in FIG. 1, in which the two ovaries are indicated by numerals 101 and 102. An illustrative example of a self-scan protocol adapted for use by an unskilled individual, which is suitable for use with the device of the invention, is the stepwise standardized approach to monitoring the follicles and the thickness of the endometrium, described by Abuhamad, Alfred, et al. (Ultrasound in obstetrics and gynecology: a practical approach, 2014). This protocol is applied in three simple steps: 1) The user holds the transducer straight in to take the first image of the uterus; 2) The user moves the transducer to the left to image the left ovary; 3) The user moves the transducer to the right to image the right ovary. A detailed illustration of this procedure is provided in FIG. 10 (a-c) of WO2023026272. When imaging the endometrium, measurements are taken in the long axis or sagittal plane, ideally on transvaginal scanning, with the entirety of the endometrial lining through to the endocervical canal in view.

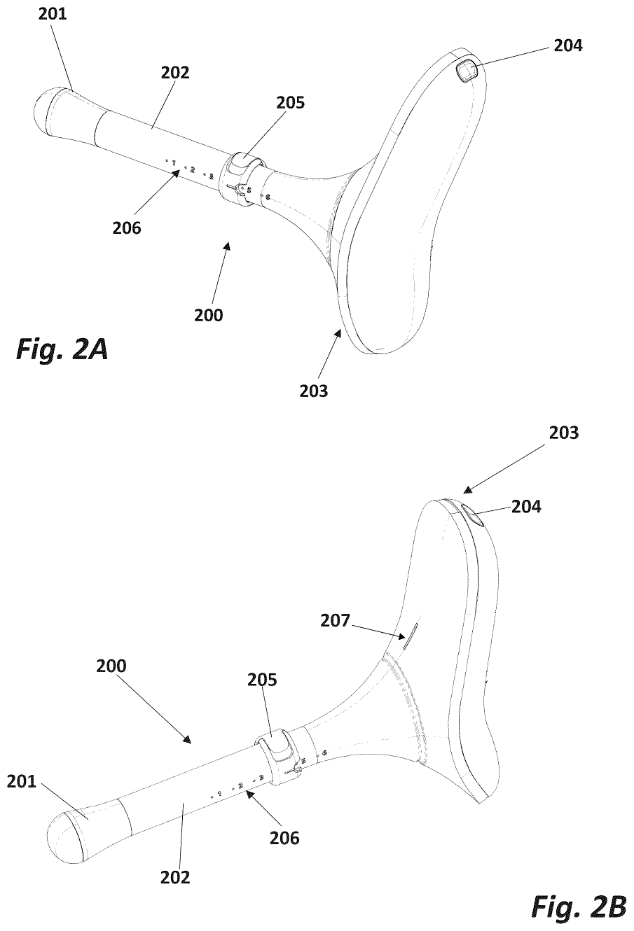

FIG. 2 (A and B) shows a device 200 according to one embodiment of the invention, viewed from two different directions. Except for the modifications made for the purposes of the present invention, the device of FIG. 2 is similar to that described in detail in WO 2024/116175 (PCT/IL2023/051218), the whole specification of which is incorporated herein by reference. The device has an ultrasound probe housed in tip 201 located at the distal end of the device and of the elongated member 202 thereof, which is to be inserted in the vagina for ultrasound scanning. A handle or grip 203 is the portion the user holds during scanning. Grip 203 may hold various components of the system, such as communication elements, image processing, and power supply, which are not discussed herein in detail for the sake of brevity since they do not influence the mechanical elements of the invention. It may also contain electronic components of the timing component according to the invention. Similarly, elongated member 202 may contain wiring or wireless components to transmit data generated by the ultrasound probe to the handle or an external device and to receive data from an external device such as, for instance, a smartphone or the like. In some embodiments, i.e., particularly in a wireless device, a pressure switch 204 is provided to power the device on and allow for ultrasound scanning. In some other embodiments, when the device is connected by wires to a smart device, such as a smartphone, no internal battery is needed in the device, which is controlled by an app running on the smart device and powered by the smart device's battery. A depth-limiting element 205 is also shown in FIG. 2, which may be used alone or in conjunction with scale 206, as further discussed in WO 2024/116175. Moreover, in some embodiments, a pressure sensor is located at the distal tip of the device. Pressure readings allow the user to determine whether the device has been introduced at a depth greater than advisable and, in some embodiments, to alert the user if the tip of the device is applying undesirable pressure. This may happen, for instance, even though the user has employed a depth-limiting element if the original depth set was incorrect or if the element has moved inadvertently. Of course, as will be apparent to the skilled person, any other suitable method can be employed, such as light measurements, distance measurement using ultrasound, and any other proximity sensor instead of a pressure sensor.

In this particular exemplary embodiment, the timing component uses a light indicator 207 to alert that the device's status is “ready for scanning.” The light indicator may, for example, be red as long as the timing component has not determined that the next scan should be performed and turn green when the time is ripe. Of course, more active alerts can be provided, e.g., by turning on an alarm in the app associated with the kit, sending a text or email message to the user, displaying an alert on the portable device associated with the ultrasound apparatus, etc.

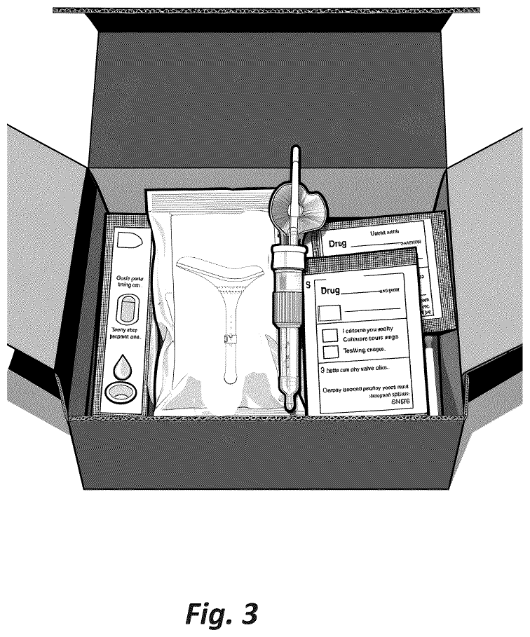

FIG. 3 illustrates a typical kit that will contain a device as shown in FIG. 2, drugs to be taken and/or injected by the subject, and test kits such as for saliva and urine.

As will be appreciated by the skilled person, the invention provides a significant step forward in the IVF process, increasing the chances of success and reducing cost as well as hassle for the medical team and the patient alike.

1. A fertility kit comprising a fertility drug and dedicated ultrasound apparatus adapted to monitor the development of a patient's follicles, wherein the apparatus comprises a timing component suitable to suggest personalized monitoring times.

2. The fertility kit of claim 1, wherein the timing component comprises a processor configured to receive data relative to the drugs administered to the user and to generate an indication of the timing of the next desirable ultrasound scan based at least on said data.

3. The fertility kit of claim 2, wherein the timing component's processor is provided in the ultrasound device.

4. The fertility kit of claim 2, wherein the timing component's processor is provided in a smart device associated with the ultrasound device.

5. The fertility kit of claim 1, further comprising ancillary tests suitable to provide information regarding the ovulation status of a subject.

6. The fertility kit of claim 5, wherein the ancillary tests are selected from saliva and urine.

7. An application adapted to run on a smart device, associated with the kit of claim 1, wherein the smart device is in communication with the dedicated ultrasound device via a network component provided therein.

8. A system for improving the precision of the timing of ultrasound scans during an IVF preparatory process or an egg preservation process, comprising:
a) a fertility kit comprising a fertility drug and dedicated ultrasound apparatus;
b) an application adapted to run on a smart device and to be in communication with the ultrasound apparatus;
c) a smart device provided with communication components adapted to exchange data with an ultrasound device and with remote computer systems.

9. The system of claim 8, wherein the smart device and the ultrasound device communicate over Wi-Fi or Bluetooth.
